(19) United States
(12) Patent Application Publication
(10) Pub. No.: US 2013/0017944 A1
(43) Pub. Date: Jan. 17, 2013

(54) SOLID CATALYTIC COMPOSITIONS BASED ON MESOPOROUS ORGANIC MATERIALS

(71) Applicants: Philippe Mesini, Sand (FR); Thi Thanh Tam Nguyen, Lure (FR); Francois-Xavier Simon, Levallois Perret (FR); Nawel Souad Khelfallah, Francfort Sur Le Main (DE)

(72) Inventors: Philippe Mesini, Sand (FR); Thi Thanh Tam Nguyen, Lure (FR); Francois-Xavier Simon, Levallois Perret (FR); Nawel Souad Khelfallah, Francfort Sur Le Main (DE)

(73) Assignees: UNIVERSITE DE STRASBOURG, Strasbourg (FR); CENTRE NATIONAL DE LA RECHERCHE SCIENTIFIQUE ( C.N.R.S.), Paris (FR)

(21) Appl. No.: 13/390,857

(22) PCT Filed: Aug. 17, 2010
(86) PCT No.: PCT/FR2010/051721

§ 371 Date: Oct. 2, 2012

(30) Foreign Application Priority Data
Aug. 17, 2009 (FR) 09 55684

(51) Int. Cl. B01J 31/06; C02F 1/00; C07C 253/30; B01J 35/10

(52) U.S. Cl. 502/5; 502/159; 210/749; 558/374

(57) ABSTRACT

The present invention relates to the preparation of a solid catalytic composition based on a functionalized porous organic material, wherein: (A) organogelator compounds are self-assembled, within a medium comprising organic monomers, in the form of fibrillar structures having a diameter ranging from 10 nm to 100 nm; and then (B) the monomers are then polymerized; and then (C) the organogelator compounds are extracted from the polymer material, thereby obtaining a porous polymer material (M⁰), wherein the monomers bear reactive R functions, for which the presence is sought on the material; or said reactive R functions in protected form; or functions able to allow grafting of said reactive R functions on the polymer materials; and wherein, following step (C), (D) all or part of the functions present at the walls of the mesopores of the polymer material (M⁰) are converted where appropriate into reactive R functions and a composition is recovered comprising a porous polymer material (M) bearing the reactive R functions, as a catalytic composition.

The present invention relates to a novel type of solid catalytic composition which appears in the form of a mesoporous solid which may in particular be used in an aqueous medium.

By “mesoporous solid”, is meant in the sense of the present description, a porous solid, the pores of which, so called “mesopores”, have at least one characteristic dimension comprised between 2 and 250 nm, typically less than 100 nm and notably between 2 and 50 nm, for example a material comprising pores or hollow channels with a diameter comprised in these ranges of values.

A mesoporous solid composition intended for catalysis has a so-called “open” porosity, i.e. at least one portion of the surface of its mesopores is accessible by chemical species (as opposed to a closed porous structure, where the pores are included in a solid matrix without any possible exchange with the outside.) A mesoporous nature associated with an open structure in particular gives the material a high specific surface area, allowing substantial contacts with the outer medium. Materials having a pore size of more than 20 nm are also particularly of interest for facilitating the transport of material within the pores on the one hand and for allowing grafting of macromolecules, such as enzymes or proteins on the other hand.

On this exchange surface, and in particular on the surface of its mesopores, a mesoporous solid intended to play the role of a catalyst includes surface functions giving it a catalytic nature. The notions of “catalytic” composition or nature, in the sense of the present invention, as well as the terms of “catalyst” or of composition or function capable of “catalyzing” a chemical reaction are understood in their broadest sense, i.e. a composition is considered as a catalytic composition, or providing a so-called catalytic effect, if it is able to promote a chemical reaction where it does not intervene as a reagent, this composition preferably being unchanged at the end of the chemical reaction. In other words, in a sense of the present description, a catalyst in a sense of the present description is understood as any composition able to promote a chemical reaction when it is introduced within a reaction medium, and in that it either plays or not a role of a catalyst in the strictest sense of the term.

In catalysis, mesoporous solids prove to be particularly of interest insofar that the contacting of the reagents and of the surface functions of the catalyst is carried out within the confined environment of the mesopores, which may notably promote contact between the reagents, increase the efficiency of the catalysis, or even allow reactions which could not be contemplated under other conditions.

To this day, known mesoporous catalysts are essentially if not exclusively solids of an inorganic nature, typically based on mineral oxides, generally based on silica, alumina, or titanium oxide of the type of those described for example in J. Am. Chem. Soc., Vol. 114, pp. 10834-10843 (2002), Science, Vol. 279, pp. 548-552 (1998), or Nature, Vol. 359, pp. 710-712 (1992).

Although these inorganic catalysts have proved their worth, they suffer from a certain number of drawbacks, to begin with that their catalytic surface functions are generally difficult to modify, and that it is consequently difficult to modulate their catalytic activity. In fact, inorganic compounds adapted to the synthesis of mesoporous catalytic solids are in limited number and functionalization of these materials not affecting the mesostructure often proves to be delicate to achieve.

Moreover, inorganic mesoporous catalysts generally have rather poor mechanical strength. Friable and brittle, they are quasi exclusively used in granular or powdery form, which reduces their possible modes of application and makes their recycling delicate. Further, when they are used in a liquid medium, notably in aqueous media, they tend to break up over time, which makes them very poorly adapted for example to the treatment of a continuous aqueous flow, which tends to erode these catalysts over time.

An object of the present invention is to provide mesoporous catalysts having the advantages of the aforementioned inorganic mesoporous catalysts but which do not have their drawbacks. In particular, the invention aims at providing mesoporous solid catalysts, the functionalization of which may be adapted to a great extent and which have a higher mechanical strength than those of inorganic mesoporous catalysts, allowing inter alia their application in an aqueous medium.

For this purpose, the present invention proposes a novel type of mesoporous solid catalyst, i.e. a material based on a mesoporous organic polymer, the surface of the pores of which is a bearer of functions able to catalyze a chemical reaction.

Organic polymer materials having a mesoporous structure have of course been described in a few rare scientific articles. However, the specific polymers contemplated in the articles have not been contemplated as potentially useful materials as catalysts and for good reasons: to the knowledge of the inventors, the mesoporous polymer materials which have been the subject of publications do not have surface functions giving them a catalytic nature. On this subject, mesoporous polymers without any catalytic nature were described in Macromol., Vol 21, pp. 274-2761 (1988), which are prepared by self-organization of polymers comprising immiscible blocks (organization by phase segregation according to a hexagonal cylindrical phase or the like), and then by degradation of one of the blocks in order to generate mesoporosity in the material. Moreover, for example in Langmuir, Vol 21, pp. 9322-9326 (2005) or in J. Am. Chem. Soc., Vol. 129, pp. 3788-3789 (2007), the preparation of organic mesoporous solids was described by polymerization of a mixture of monomers (divinylbenzene, ethylene diacrylate) including so-called organogelators which self-associate within the mixture of monomers in the form of fibers or tubes, which leads to the formation of a polymer integrating these fibers or tubes, which are then discharged out of the polymer, thereby leaving in the polymer, mesopores (channels) corresponding to the imprint of the fibers initially formed by the organogelator.

More generally, there exist few techniques known to this day with which porous organic polymers functionalized within their pores with potentially useful groups in catalysis may be obtained. As a rare example of a technique of this type, mention may be made of the polymer synthesis from Acc. Chem. Res. Vol. 34, pp. 973-980 (2001), which uses amphiphilic monomers of lyotropic nature, capable of forming hexagonal cylindrical phases, which leads to polymers with a porous structure and bearing polar groups (the polymer having the structure of the cylindrical phase formed by the lyotropic monomers). In addition to the fact that this technique is limited as regards the functions present in fine inside the pores (these groups are necessarily polar), it is especially limited to the obtaining of microporous polymer (very small pore diameter of less than 2 nm) and cannot be transposed to the synthesis of polymers having pores with a greater size, in other words, this technique cannot be exploited for synthesizing functionalized mesoporous polymers.

The present invention conversely provides means for obtaining organic materials having a mesoporous nature on the one hand and for which the functionality of the walls may be modulated to a very great extent.

More specifically, according to a first aspect, the object of the present invention is a method for preparing a solid catalytic composition based on a mesoporous polymer material, for which the walls of the mesopores bear reactive functions R able to catalyze a chemical reaction, which comprises the following successive steps:

(A) within a medium comprising organic monomers, self-assembling of organogelator compounds is achieved in the form of fibrillar structures having a diameter from 10 nm to 100 nm, whereby a gel is formed, comprising said fibrillar structures dispersed in a dispersive medium comprising the monomers; and then

(B) polymerization of the monomers present in the dispersive medium of the thereby prepared gel is carried out, whereby a polymer is formed around said fibrillar structures; and then

(C) the organogelator compounds are extracted out of the thereby prepared polymer material, whereby a porous polymer material (M⁰) is obtained,

wherein the organic monomers used bear functions R′, not engaged into the polymerization reaction of step (B), whereby the polymer material obtained at the end of step (C) comprises these functions R′ at least at the walls of its mesopores, these functions R′ being:
reactive functions R able to catalyze a chemical reaction, the presence of which is sought on the material; or
said reactive functions R in a protected form; or
functions R″, able to allow grafting of said reactive functions R on the polymer material obtained at the end of step (C), or else such functions in a protected form,
   and then

(D) if necessary all or part of the functions R′ present at the walls of the mesopores of the polymer material (M⁰) from step (C) are converted into reactive functions R, and a composition is recovered comprising a porous polymer material (M) bearing reactive functions R within its mesopores, as a catalytic composition.

According to another aspect, the object of the invention is also a solid catalytic composition based on a mesoporous polymer material, for which the walls of the mesopores bear reactive functions R able to catalyze a chemical reaction, which have the specific characteristics obtained by applying the aforementioned steps (A) to (D).

With the work accomplished by the inventors within the scope of the present invention it was now possible to demonstrate that by applying steps (A) to (D) above, functionalized mesoporous organic materials may be obtained in a simple and relatively inexpensive and efficient way and this with a very large functionalization modularity of the surface of the mesopores, by which it is possible to attain directly and very simply a very wide range of organic catalysts which may be used over an extended panel of chemical reactions.

Indeed, it should first of all be noted that in the method of the invention, mesoporosity is obtained very easily, i.e. simply by carrying out polymerization of the monomers in the presence of organogelator compounds which in the polymerization medium, self-associate in the form of fibrillar (tubular) objects with mesoscopic dimensions, these objects playing the role of a “template” during the synthesis of the polymer and then being removed at the end of the polymerization, which leads to obtaining in the final material, mesopores corresponding to the imprint of the fibrillar objects formed by the organogelators. In addition to their ease of application, the applied organogelators within this framework generally have the advantage of being able to be recycled, which shows a notable advantage of the method, which is expressed in terms of reduced process costs, and which makes the method notably adapted to an application of the method on an industrial scale. The method of the invention within this framework has an unquestionable advantage as compared with the presently known methods for synthesizing inorganic mesoporous materials, which apply organic molecules as a template (surfactants being associated in a phase of the hexagonal type typically). Indeed, in the case of organic materials generally sensitive to washing operations, the template used is generally removed by calcination, which prevents any possibility of recycling.

Moreover, the application of other steps between (A) to (C) allows the application of a very large number of functions R′, themselves able to be modified in step (D) in a still more extended range of reactive functions R, which opens the road to functionalization modularity incomparable with the much more restricted one, provided by inorganic catalysts.

Further, the materials obtained according to steps (A) to (D) have mechanical properties which are substantially those of the organic polymers which make them up, generally independently of the R functions which they bear. In any case, the materials obtained according to steps (A) to (D) notably have a higher mechanical strength than the inorganic mesoporous catalysts, and they do not have the friable and brittle nature of the latter. Further, they may be applied in liquid media where the polymer is not soluble, notably in an aqueous medium, without leading to erosion phenomena encountered with inorganic mesoporous catalysts.

Further, unlike the inorganic mesoporous catalysts, which are quasi systematically applied in granular or powdery form (often including very fine particles), the mesoporous organic catalysts according to the invention may be used in more macroscopic forms. Indeed, the gel prepared in step (A) may be shaped with very high modularity before the polymerization of step (B), whereby varied forms may be obtained for the mesoporous material (M) obtained at the end of step (D). For example, the gel prepared in step (A) may be introduced into a mold, whereby the material (M) obtained in fine appears as a molded mesoporous mass part having the shape of the mold used. Alternatively, taking into account its relatively large viscosity, the gel prepared in step (A) may be deposited as a film, on various supports, with possibility of controlling the thickness of the film, by which it is for example possible to obtain the catalytic material (M) as a surface coating on different types of substrates, notably in the form of an internal reactor coating. This possibility of shaping the catalysts, associated with their capability of being used in a liquid medium proves to be particularly adapted to recycling of the catalyst (shaping into the form of a bulk object or particles with sufficiently great size (milimetric beads for example) facilitates extraction of the catalyst which may then be optionally subject to washing steps without any risk of erosion).

Different characteristics and preferential embodiments of the method of the invention will now be described in more details.

Generally, the method of the invention is carried out by using monomers bearing functions R′ as defined above, associated with organogelators capable of forming fibrillar objects within a medium comprising these monomers.

According to a particular embodiment, the functions R′ present on the monomers used in step (A) of the method are reactive functions R, the presence of which is sought on the final material in protected form. By “protected form” of a reactive function R, is meant in the sense of the present invention, a function which is capable of being easily converted into the function R, generally in a single step, by a reaction which may be applied during step (D) without being detrimental to the integrity of the structure of the porous polymer (M⁰) prepared in step (C). In the sense of the present description, such a protected form in particular includes precursor groups of the functions R′. In the case when the functions R′ present on the monomers of step (A) are such reactive functions R in protected form, the step (D) comprises a reaction for deprotecting all or part of the functions R′ into the sought reactive functions R. According to this embodiment, the functions R′ may for example be ester functions and the step (D) then includes a deprotection (conversion) reaction of all or part of these esters into reactive carboxylate functions —COO⁻ typically by treating the polymer material (M⁰) obtained at the end of step (C) with a base, for example a soda or potash solution.

According to another embodiment of the invention, the functions R′ present on the monomers used in step (A) are generally non-reactive functions as such, giving the possibility of attaching onto the material (M⁰) such reactive functions R, in a grafting step or optionally in several successive synthesis steps. In this second scenario, in step (D), the functions R are attached onto the surface of the mesopores of the material (M⁰) by means of these adhesion functions R′, generally by reaction of the material (M⁰) with compounds bearing both R′″ functions capable of forming a covalent bond with the R′ functions; and of R functions, optionally in protected form in particular if the latter are otherwise capable of reacting with the functions R′ or and then, if necessary, deprotection of the R functions. According to this embodiment, the R′ functions present on the monomers used in step (A) may for example be ester functions. In which case, in step (D), these functions are at least partly deprotected into carboxylic acid or carboxylate functions, and the R functions are then introduced, typically by reaction of the material, (M⁰), with a compound bearing an amine or alcohol function (preferably including a group >N—H or —OH) and at least one R function. Alternatively, the R′ functions present on the monomers used in step (A) may be protected amine functions which, in step (D) are at least partly deprotected into primary or secondary amine functions, and then the R functions are introduced, typically by reaction of the material (M⁰) with a compound bearing a carboxylic acid function, an acid chloride (sulfonyl chloride for example) function or/and of at least one R function, or else more generally by reaction with a reagent of formula R-A, wherein A is a leaving group, for example a group of the halide or mesylate type.

As monomers particularly well adapted to a use within the scope of step (A) of the method of the invention, which proves to be notably of interest in the embodiments of both preceding paragraphs, mention may notably be made of monomers bearing ester functions, which lead to the formation in step (B) of polymers bearing these same ester functions as R′ functions. (Meth)acrylic esters of general formula H₂C═CR¹—COOR², prove to be particularly interesting within this scope, wherein R¹═H or CH₃ and wherein R² is a hydrocarbon chain (optionally either totally or partly cyclized) preferably including 1 to 6 carbon atoms and optionally bearing a (meth)acrylic ester group. Preferably, in step (A), a mixture of difunctional monomers is applied, i.e. bearing two polymerizable (meth)acrylic groups, optionally mixed with monomers bearing a single polymerizable (meth)acrylic group. Difunctional (meth)acrylic ester monomers particularly suitable for applying the method of the present invention are esters of (meth)acrylic acid and of a diol, typically fitting the formula H₂C═CR¹—COO-A-OOC—R¹—C═CH₂ wherein R¹═H or CH₃ and wherein R² is a hydrocarbon chain, preferably an alkylene group, preferably from 1 to 6 carbon atoms, such as for example ethylene diacrylate (CH₂═CH—COO—CH₂—CH₂—OOC—CH═CH₂) or butylidene diacrylate (CH₂═CH—COO—(CH₂)₄—OOC—CH═CH₂), or else further (meth)acrylates including protected amines, such as (CH₂═CR¹—COO—(CH₂)ₙ—NH—CO^{t}Bu, or CH₂═CR¹—COO—(CH₂)ₙ—N—CO^{t}Bu—(CH₂)ₙ—OOC—CR¹═CH₂).

The invention cannot however be limited to the application of the (meth)acrylic monomers described in the previous paragraph and many other types of monomers thereby lend themselves to the application of the invention, in particular many photopolymerizable unsaturated monomers such as mixtures of monomers of the CH₂═CH—Ar—CH═CH₂/CH₂═CH—Ar—Y type, wherein Y is an ester group (typically of formula —COOR³ wherein R³ is a hydrocarbon chain, for example an alkyl), an alcohol group or a halogen.

Regardless of the nature of the monomers applied in step (A), the latter are applied in this step in the presence of organogelator compounds, under conditions where these organogelators form in the monomer mixture, fibrillar structures having a diameter from 10 nm to 100 nm, which used as a template in the method of the invention, lead to calibrated and controlled mesoporosity in the material (M) obtained at the end of the method.

By “organogelator compounds” or “organogelators” of a given liquid medium, is meant in the sense of the present description, a set of identical organic molecules generally not polymers, or a mixture of several generally non-polymeric organic molecules, which, when they are introduced into said liquid medium are capable, at least under certain conditions of concentration and temperature, of establishing between them physical interactions so that they self-associate, generally by means of weak reversible bonds (typically hydrogen bonds) so as to form within the apolar medium, three-dimensional supramolecular structures associating several molecules, which generally leads to gelling of the medium. For more details concerning organogelators, reference may notably be made to Chem. Rev. Vol. 97, pp. 3133-3159 (1997) or else to the textbook “P. Molecular gels Materials with self-assembled Fibrillar Network”; Springer: Dordrecht, The Netherlands (2006).

The organogelators used within the scope of the present invention are molecules which are capable of forming associations of the fibrillar type inside the medium of step (A). By “fibrillar structure” is meant a structure with a cylindrical or substantially cylindrical morphology, characterized by a length along a longitudinal axis and a radius perpendicular to this longitudinal axis with a length to radius ratio preferably greater than 10:1, more preferentially greater than 20:1 and even more preferentially greater than 40:1, or even 50:1. The organogelators used within the scope of step (A) are molecules capable of forming fibrillar structures with a diameter of 10 and 100 nm, for example from 15 to 80 nm, notably from 20 to 50 nm. The length of these fibrillar structures is preferably at least 200 nm and more preferentially at least 300 nm, typically of the order of 400 nm to 5 microns.

The organogelators used within the scope of the present invention are moreover compatible with the monomers used. Thus, organogelators which are dissolved in or precipitate from the monomers used should notably be banned. Suitable organogelators within the scope are in a non limiting way the organogelator systems used in the aforementioned articles of Langmuir, Vol 21, pp. 9322-9326 (2005) and J. Am. Chem. Soc., Vol. 129, pp. 3788-3789 (2007).

According to a preferential embodiment of the invention, the medium of step (A) is a polar medium and the organogelators compounds used are compounds capable of self-associating in an apolar medium. Alternatively, the medium of step (A) may comprise apolar compounds, in which case the organogelator compounds used are compounds capable of self-associating in an apolar medium.

According to an embodiment particularly well adapted to the application of steps (A) to (C), and this notably when the monomers used are meth(acrylic) esters or apolar monomers, it is of interest that the organogelator compounds applied in step (A) be molecules of decyl 3,5-bis-(5-hexylcarbamoyl-pentyloxy)benzoate (a compound also called BHPB), fitting the following formula:

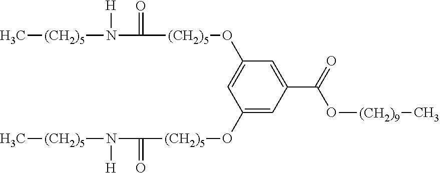

which is a compound which in an apolar medium forms in a known way, self-associations through hydrogen bonds which lead to the formation of nanotubular type structures having a controlled diameter, not very polydispersed, of the order of 30 nm and a length of 400 nm to a few microns, notably observable with electron microscopy and small angle neutron scattering.

More generally, it is possible to apply as an organogelator in step (A) analogue compounds from the aforementioned family of BHPBs, fitting the following formula (I):

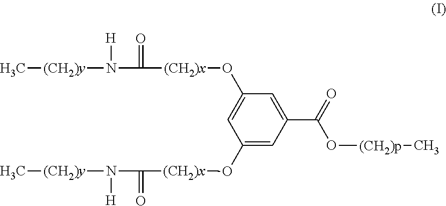

wherein:
x is an integer ranging from 2 to 7,
y is an integer ranging from 1 to 10, such that the sum (x+y) ranges from 8 to 12;
z is an integer greater than 7, generally between 8 and 16, for example between 8 and 12.

Preferred compounds of formula (I) are those wherein x=y=5 and wherein z=8, 9, 10, 11 or 12.

The compounds fitting the general formula (I) lead to the formation of nanotubes in an apolar medium, these nanotubes typically having a diameter of the order of 22 to 40 nm.

As organogelators in step (A), it is possible to apply other organogelator compounds able to form the sought fibrillar structures in the medium of step (A), for example:
the combination of sodium bis-(2-ethylhexyl)sulfosuccinate (AOT) and 4-chlorophenol described in Langmuir, Vol 21, pp. 9322-9326 (2005); or
the peptide derivatives described in Chem. Commun. 738-739 (2002) and used in Langmuir, 24, 9795-9803 (2008).

Depending on the nature of the organogelator compounds applied in step (A), a specific texturation of the material is obtained, with in particular a diameter of mesopores characteristic of the organogelator compounds used. It is thus possible to modify the structure of the material (M) prepared according to the invention in a fine way, and in particular modulate the diameter of its mesopores by a wisely selecting the organogelator compounds used.

Depending on the organogelators used, the obtaining of fibrillar structures in step (A) is possibly subordinated to specific conditions of concentrations and/or of temperature. For example, in order to obtain fibrillar structures (nanotubes) in the medium of step (A) from BHPB (and more generally from compounds of formula (I)), it is generally desirable that the BHPB/monomers mass ratio be greater than 0.05:1, the porosity and the specific surface area of the material (M) in fine increasing with this ratio. In order not to introduce too large porosity which may be detrimental to the mechanical properties of the material (M) otherwise, it is most often preferable that the BHPB/monomers mass ratio remain less than 2:1. In order to obtain high porosity and good mechanical properties, this mass ratio is preferably comprised between 0.5:1 and 1.5:1, typically of the order of 1:1. A similar problem is encountered with the whole of the organogelators. It is within the competences of one skilled in the art to adapt case by case, the concentration of organogelators to be applied in order to obtain the desired porosity and mechanical strength.

Moreover, it should be noted that the fibrillar structures based on BHPB (or more generally on the compounds of formula (I)) are spontaneously formed at room temperature but disappear at a limiting temperature, a so-called “gelling temperature”, which depends on the concentration of organogelators in the medium (this temperature is for example 58° C. for a 5% mass content of BHPB). Fibrillar structures based on BHPB are typically made by hot dissolution of BHPB into the monomer mixture and then by cooling the medium down to room temperature (typically 25° C.). Thus, when BHPB is used as an organogelator, it is desirable that the whole of the steps (A) and (B) be conducted below this critical temperature. As this remark is valid for a number of other organogelator systems, it most often proves to be preferable that in the method of the invention, steps (A) and (B) be conducted at room temperature as soon as the fibrillar structures are formed so as not to risk affecting the integrity of these structures.

In particular for this purpose, it is preferable that the polymerization of step (B) be conducted by photo-initiation, which allows application of step (B) at a low temperature, typically room temperature. Such an application of step (B) by photopolymerization may be achieved according to any embodiment known per se to one skilled in the art. Typically, a photo-activatable polymerization initiator i.e. a compound capable of forming free radicals when subject to irradiation (typically in the UV range), is added to the mixture of polymers, and photopolymerization is achieved by subjecting the mixture to this irradiation.

Once the polymer is formed in step (B), the extraction of organogelator compounds which is performed in step (C) may be carried out according to any means known per se it being understood that this extraction should not be of a nature affecting the synthesized polymer, and in particular it should not inhibit the possibility of obtaining in fine the sought reactive functions R on the surface of the mesopores of the polymer. For this purpose the extraction of the organogelator compounds in step (C) is generally conducted by washing, typically by using a solvent capable of dissociating the fibrillar structures formed by the organogelators, while preferably conducting several successive washings of the polymer, preferably by soaking in successive washing baths. Such an embodiment generally allows quantitative extraction or even substantial extraction (typically in an amount of more than 90%, and generally in an amount of at least 95%) of the organogelator compounds out of the mesopores of the polymer formed in step (B) without affecting neither the structure of the polymer nor the organogelator compounds which are easily recoverable from washing solvents, for example by simple evaporation of the solvent. According to an embodiment, the thereby extracted organogelator compounds are recycled, notably so as to be again applied in a step (A) for forming a material according to the invention, after optional purification of the organogelators. This recycling possibility of the organogelators is again an advantage of the method of the invention, which notably has repercussions in terms of reduced production costs.

For example, when BHPB is used as an organogelator compound, step (C) may typically be conducted by washing the polymer stemming from step (B) in a solvent capable of dissociating the hydrogen bonds associating the molecules of BHPB in the nanotubes formed by these organogelators. A dissociating solvent suitable for this purpose is for example chloroform which allows substantial extraction of the organogelators (typically, an immersion of the polymer in three chloroform washing baths leads to an extraction of more than 95% of the BHPB out of the polymer (typically of the order of 99%), the thereby extracted BHPB may be recovered and re-used, for example as an organogelator agent in step (A) for synthesizing another polymeric mesoporous material according to the invention.

Regardless of their exact preparation method, the solid catalytic compositions of the present invention are based on a mesoporous polymer material, where the mesopores typically appear as channels with a calibrated and controlled diameter comprised between 5 and 150 nm, most often between 10 and 100 nm, more generally between 15 and 80 nm, notably between 20 and 50 nm (typically about 30 nm when BHPB is used as an organogelator).

Moreover, a catalytic composition according to the invention most often appears as a hard and mechanically resistant polymer resin. With these mechanical properties, it is possible to reduce the composition in particles with millimetric dimensions without affecting the structure of the mesopores of the material (M) and without leading to the formation of fine particles of the type of those present in compositions based on much more friable inorganic mesoporous materials.

Depending on the mode of application of steps (A) and (B), a composition according to the invention may appear:
as a massive polymer part, for example a molded polymer part such as a hollow tube, a container, or a plate (to do this, the gel prepared in step (A) is placed in an adequate mold before polymerization of step (B)); or
as a catalytic coating deposited on all or part of a support, for example on the inner face of a duct or of a container (for this purpose, the gel prepared in step (A) is deposited on a support prior to the application of step (B)); or
as particles with millimetric dimensions (for example with an average diameter of the order of 0.5 to 10 mm).

The compositions obtained according to the method of the invention may be used as solid catalytic compositions for promoting diverse chemical reactions.

Within this scope, the compositions obtained according to the aforementioned steps (A) to (D), prove to be most particularly of interest for use in a liquid medium, notably in an aqueous medium.

Within this scope, a composition according to the invention may appear as sufficiently divided particles in order to provide an exchange surface area suitable for catalysis, but with a sufficiently large size in order to allow easy separation of the obtained products and of the catalyst, for example as particles with dimensions of the order of 1 mm. This possibility of obtaining the catalyst as easily separable particles is a notable advantage as compared with inorganic catalysts in powdery form, which inevitably include very fine particles (micron or submicron particles) which may contaminate the obtained products. According to an embodiment which may be contemplated, particles with a composition according to the invention may be agglomerated (notably by sintering) by which porous materials with the highest specific surface area may be obtained. Moreover, easy extraction of the catalyst allows washing and recycling of the catalyst at the end of the catalysis of the reaction, generally without any substantial loss in catalytic efficiency.

Moreover, a composition according to the invention in the form of a tube or of a container or as a catalytic coating on the internal surface of a duct or of a container may be used for continuously treating a liquid flow, notably an aqueous flow, during transport or transfer. Within this scope, the compositions of the invention may for example be used for treating waste water or industrial chemical effluents.

Different aspects and preferential characteristics of the invention are illustrated in the exemplary embodiments hereafter.

### EXAMPLE 1

### Preparation of a Catalytic Composition According to the Invention (Resin R)

0.2 g of BHPB (decyl 3,5-bis-(5-hexylcarbamoyl-pentyloxy)benzoate) and 0.2 g of a 3% solution of Irgacure 651 photoinitiator (2,2-dimethoxy-1,2-diphenyl-ethanone) were dissolved in ethylene diacrylate H₂C═C—C═O—CH₂—CH₂—C═O—C═CH₂ with a BHPB/ethylene diacrylate mass ratio of 1:0.97. Dissolution was carried out by introducing the compounds into a closed container and heating the obtained mixture to 70° C., until complete dissolution of BHPB and of the photoinitiator in benzene diacrylate.

By leaving the thereby obtained solution to cool without stirring, down to room temperature (25° C.), gelling of the medium was obtained, due to the organization of BHPB in the form of nanotubes within the mixture of monomer and of photoinitiator.

By maintaining the medium at room temperature and without stirring, the thereby formed gel was subject to UV irradiation, whereby photopolymerization of the ethylene diacrylate was obtained around BHPB nanotubes present in the reaction medium, in the form of a polyacrylate resin.

In order to extract the BHPB present in the pores of the thereby achieved resin, the latter was immersed in a chloroform bath for 24 hours. The resin was removed from the bath and then again immersed twice successively in two new fresh chloroform baths for 24 hours at each time. At the end of these washing steps, a mesoporous resin was obtained, designated hereafter by R⁰.

The contents of the three washing baths were collected and the chloroform was evaporated, by which it was possible to recover a mixture of 205 g (+/−1 g) of BHPB and of Irgacure, which after purification allows recovery of 97% of the initially introduced BHPB mass. The thereby recovered purified BHPB may be recycled in order to again form a resin.

An analysis of the R⁰ resin by infrared spectrometry in an ATR mode and by NMR of the solid shows that the latter comprises less than 0.1% of the initially introduced BHPB mass, which reflects the good efficiency of the washing.

Moreover, electron micrographs of the resin R⁰ show a porous internal structure dug with homogeneous channels or having a diameter of the order of 30 nm, these channels exactly corresponding to the imprint of the BHPB nanotubes which were removed during the washing steps. An analysis of the porosity of the material (porosimetry according to the BET method) confirms the presence of narrow porosity centered on 30 nm.

The resin R⁰ was milled in the form of particles with dimensions of the order of 1 mm. The thereby milled resin was immersed for 260 minutes in a 1 mol/l NaOH solution, in a methanol/water 1:1 v/v mixture, which led to partial hydrolysis of the ester functions of the resin into carboxylate functions (which may notably be detected by Fourier transform infrared spectrometry), and the resin was then removed from the treatment bath and rinsed with water, and then with methanol by means of a Soxhlet extractor. The resin R was thereby obtained, in the form of grains with a diameter of the order of 1 mm, based on a hard and glassy material.

### EXAMPLE 2

### The Use of the Resin R of Example 1 as a Solid Basic Catalytic Composition

The resin R of Example 1 was used as a catalyst in a model reaction, i.e. the Knoevenagel condensation of benzaldehyde and ethyl cyanoacetate below:

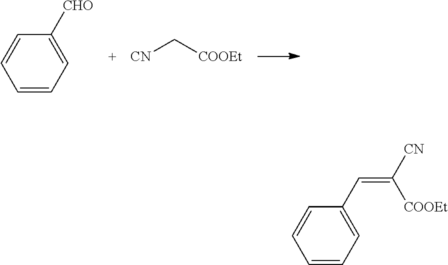

The reaction was conducted at 25° C., under the following conditions:

0.6 g of benzaldehyde and 0.9 g of ethyl cyanoacetate were dissolved in 10 ml of ethanol. To this solution, 0.158 g of resin R as obtained in Example 1, was added. Efficient catalysis of the reaction was obtained, with relatively high yield (95%) and interesting kinetics, followed by gas chromatography (a reaction completed in about 8 hours).

At the end of the reaction, the resin was recovered by simple filtration, and washed with ethanol. The thereby recovered resin was again tested under the same conditions as above. A new cycle for recovering and reusing the resin was then achieved again.

These successive recovery, washing and reuse cycles do not notably affect the catalytic properties of the resin, the kinetics and the yield of the three successive Knoevenagel reactions having proved to be substantially identical, as indicated by the table below:

TABLE 1
Tracking the formation of the reaction
product during the three cycles

| Time | Reaction product concentration in the medium | | |
|---|---|---|---|
| (min) | Cycle 1 | Cycle 2 | Cycle 3 |
| 60 | 0.16 | 0.19 | 0.15 |
| 120 | 0.33 | 0.30 | 0.28 |
| 200 | 0.45 | 0.40 | 0.36 |
| 400 | 0.51 | 0.49 | 0.46 |
| 500 | 0.59 | 0.50 | 0.49 |

As a comparison, a control was made by reproducing the experimental conditions above, but in the absence of the resin R. Two comparative tests were moreover carried out by substituting the resin R with another resin:

Control: experimental conditions of Example 2 are reproduced, but without any resin in the reaction medium

### Comparative Test 1

Experimental conditions of Example 2 were reproduced, but by replacing the resin R with the resin R⁰ of Example 1 (mesoporous resin, non-functionalized inside its pores).

### Comparative Test 2

Experimental conditions of Example 2 were reproduced, but by replacing the resin R by a resin, called R′, obtained under the conditions of Example 1 but in the absence of BHPB (non-mesoporous resin, treated with NaOH, bearing a carboxylate function at the surface)

The conversions of benzaldehyde and ethyl cyanoethate were not observed within the scope of the control and of the comparative tests 1 and 2, are less than 5% with comparable reaction times, which shows that the resins R⁰ and R′ do not have any catalytic activity, unlike the resin R according to the invention.

### EXAMPLE 3

### Formation of a Catalytic Resin by Using Protected Monomers

A resin R″ was prepared in a similar way to the resin R of Example 1, with 0.2 g of BHPB, 0.1 g of butylidene diacrylate, 0.1 g of tertbutyl acrylate and 3 mg of Irgacure 651 (2,2-dimethoxy-1,2-diphenyl-ethanone). BHPB is extracted in order to provide a mesoporous resin with a pore size of the order of 30 nm and including tertbutylester functions.

The thereby obtained resin is milled as particles with a size of about 1 mm. These particles are immersed in 5 ml of a solution of trifluoroacetic acid in dichloromethane and left without stirring for 24 hours. They are then rinsed for 24 hours in dichloromethane and then dried at 50° C. for 48 hours.

The FTIR spectrum in the ATR mode show that the thereby obtained R″ contains carboxylic acid functions. An assay of these functions shows that their amount is 1.5 mmol/g.

### EXAMPLE 4

### Grafting Reactive Functions on the Resin R

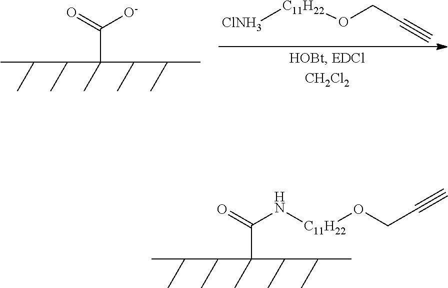

The resin R described in Example 1 is suspended (0.15 g) in a solution of 21 mg of 11-prop-2-ynyloxy-undecylammonium chloride and of 2.5 mg of hydroxybenzotriazole (HOBt) in 5 mL of dichloromethane, and then 46 mg of 1-ethyl-3-(3-dimethylaminopropyl)carbodiimide (EDCI) is added at 0° C. The suspension is stirred for 10 mins at 0° C. and then for 24 hours at 25° C. The obtained resin is filtered and washed with dichloromethane, and then with acetone and dried in vacuo. The presence of the formed amide functions is detected by FTIR. The alkyne functions introduced onto the resin may then be modified by Huisgen cycloaddition.

1-14. (canceled)

15. A method for preparing a solid catalytic composition based on a mesoporous polymer material, for which the walls of the mesopores bear reactive functions R able to catalyze a chemical reaction, which comprises the following successive steps:
(A) within a medium comprising organic monomers, self-assembling of organogelator compounds in the form of fibrillar structures having a diameter from 10 nm to 100 nm is achieved, whereby a gel is formed comprising said fibrillar structures in a dispersive medium comprising the monomers; and then
(B) polymerization of the monomers present in the dispersive medium of the thereby prepared gel is carried out, whereby a polymer is formed around said fibrillar structures; and then
(C) the organogelator compounds are extracted out of the thereby prepared polymer material, whereby a porous polymer material (M⁰) is obtained,
wherein the organic monomers used bear R′ functions, not engaged into the polymerization reaction of step (B), whereby the polymer material (M⁰) obtained at the end of step (C) comprises these R′ functions at least at the walls of its mesopores, these R′ functions being:
reactive R functions able to catalyze a chemical reaction, the presence of which is sought on the material; or
said reactive R functions in protected form; or
R″ functions, able to allow grafting of said reactive R functions on the polymer material obtained at the end of step (C) or else such functions in a protected form,
and then
(D) if necessary, all or part of the R′ functions present at the walls of the mesopores of the polymer material (M⁰) stemming from step (C) are converted into reactive R functions, and a composition is recovered comprising a porous polymer material (M) bearing reactive R functions within its mesopores, as a catalytic composition.

16. The method according to claim 15, wherein the R′ functions present on the monomers used in step (A) are reactive R functions in protected form, and wherein step (D) comprises a reaction for deprotecting all or part of the R′ functions into reactive R functions.

17. The method according to claim 16, wherein the R′ functions are ester functions and wherein step (D) includes a reaction for deprotecting all or part of these esters into reactive carboxylate functions —COO⁻.

18. The method according to claim 15, wherein the R′ functions present on the monomers used in step (A) are functions allowing the reactive functions R to be attached onto the material (M⁰) and wherein in step (D), the R functions are attached on the surface of the mesopores of the material (M⁰) by a reaction of said material (M⁰) with compounds both bearing R′″ functions capable of forming a covalent bond with the R′ functions; and R functions.

19. The method according to claim 15, wherein the monomers applied in step (A) are (meth)acrylic esters, whereby the polymer formed in step (B) is a meth(acrylic) polyester comprising ester functions as R′ functions.

20. The method according to claim 15, wherein the organogelator compounds applied in step (A) fit the formula (I) below: 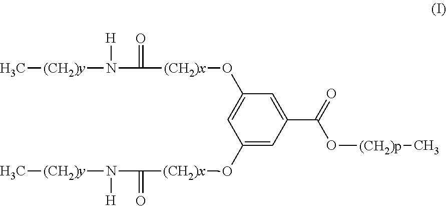
wherein:
x is an integer ranging from 2 to 7,
y is an integer ranging from 1 to 10, such that the sum (x+y) ranges from 8 to 12;
z is an integer greater than 7.

21. The method according to claim 20, wherein the organogelator compounds applied in step (A) are molecules of decyl 3,5-bis-(5-hexylcarbamoyl-pentyloxy)benzoate (BHPB) of the following formula:

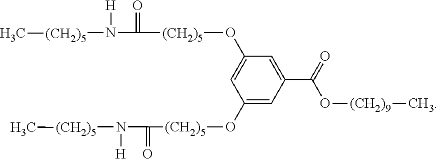

22. The method according to claim 15, wherein the polymerization of step (B) is conducted by photocatalysis.

23. The method according to claim 15, wherein, in step (C), the extraction of the organogelator compounds is conducted by washing, by using a solvent capable of dissociating the fibrillar structures formed by the organogelators, the thereby extracted organogelator compounds being advantageously recycled.

24. A mesoporous solid composition based on a polymer material, for which the walls of the mesopores bear reactive R functions able to catalyze a chemical reaction, which may be obtained according to the method of claim 15.

25. The composition according to claim 24, appearing as a massive polymer part or as a coating deposited on all or part of a support.

26. The use of a composition according to claim 24, as a catalytic composition.

27. The use of according to claim 26, wherein the composition is used in a liquid medium.

28. The use according to claim 27, wherein the composition is used in an aqueous medium.
